(19) United States
(12) Patent Application Publication
(10) Pub. No.: US 2007/0259440 A1
(43) Pub. Date: Nov. 8, 2007

(54) Measuring low levels of methane in carbon dioxide

(76) Inventors: Xin Zhou, Racho Cucamonga, CA (US); Greg Sanger, Chico, CA (US)

Correspondence Address: MINTZ, LEVIN, COHN, FERRIS, GLOVSKY AND POPEO, P.C, LA JOLLA CENTRE II, 9255 TOWNE CENTRE DRIVE, SUITE 600, SAN DIEGO, CA, 92121-3039, US

(21) Appl. No.: 11/788,757

(22) Filed: Apr. 19, 2007

Related U.S. Application Data
(60) Provisional application No. 60/793,402, filed on Apr. 19, 2006.

(51) Int. Cl. G01N 33/00

(52) U.S. Cl. 436/141

(57) ABSTRACT

Low concentrations of methane in a gas mixture containing a substantial concentration of carbon dioxide can be detected and quantified using absorption spectroscopy in the infrared spectral region. Absorption spectra can recorded using tunable diode lasers as the light source. Modulation of the laser signal and demodulation of the resultant detector response yields dependable measurements that may be conducted with very little maintenance in demanding environments.

### RELATED APPLICATIONS

The present patent application claims priority under 35 U.S.C. §119 to U.S. Provisional Patent Application Ser. No. 60/793,402, filed on Apr. 19, 2006, and entitled “Measurement of Low Levels of CH₄ in CO₂”, the entire disclosure of which is incorporated by reference herein.

### TECHNICAL FIELD

The subject matter disclosed herein relates to measurements of methane concentrations in gas mixtures containing carbon dioxide.

### BACKGROUND

Carbon dioxide is used in a number of industrial and commercial processes in which accurate, reproducible, and rugged measurements of methane impurities may be needed. These applications include semiconductor processing methods that make use of supercritical carbon dioxide as a more environmental benign alternative to conventional solvents. In addition, carbon dioxide may be used for carbonation of water and other liquids in the beverage industry, for refrigeration and cooling, as an inert gas in chemical processes or as a shield gas in TIG welding, fire extinguishers, in oil fields as a pumped-in gas for oil extraction and maintaining pressure in the drilled formation, as an additive to medical oxygen as a respiration stimulant, as a propellant in aerosol cans, and for sequestration of coal beds

The use of currently available techniques for characterizing methane concentrations in gas mixtures containing carbon dioxide for routine measurements in field applications can be hindered by various factors. For example, maintenance and calibration issues can be cumbersome and costly. In addition, calibration difficulties, detector drift, and somewhat slow response and recovery times can negatively impact the use of such techniques.

Currently available detection methods for methane in carbon dioxide include non-dispersive infrared spectroscopy (NDIR) which may suffer from lower detection limitations as well as absorption band overlap, laser systems for gas detection that use differential absorption of radiation backscattered from topographic targets, and thermal conductivity detectors (TCD) used in concert with a gas chromatograph to separate individual compounds in a gas based on their partitioning between a mobile and a stationary phase. which suffers from cycle time

### SUMMARY

In one aspect, trace amounts of methane in carbon dioxide backgrounds are detected by directing a beam of light at a selected wavelength through a gas mixture that includes carbon dioxide and methane. The selected wavelength coincides with a methane absorption feature that is resolvable from an absorption background due to carbon dioxide. An absorption at the selected wavelength I quantified in the gas mixture, and a methane concentration in the gas mixture is determined based on the quantified absorption and a calibration function.

In another interrelated aspect, an apparatus includes a laser light source that emits at a selected wavelength that coincides with a methane absorption feature that is resolvable from an absorption background due to carbon dioxide, a sample cell to contain a gas mixture including trace methane in carbon dioxide and having a path length over which light from the laser light source may be absorbed by the gas mixture, a photodetector positioned to quantify an intensity of light traversing the path length and to output a direct current data signal based on the quantified intensity; and a microprocessor configured to receive and interpret the direct current signal from the photodetector and to determine the methane concentration in the gas mixture based on the direct current data signal.

In optional variations, the gas mixture may be contained within a sample cell that provides the path length. The absorption at the selected wavelength may be quantified with a photodetector that provides a detector output signal to a microprocessor. Light may be generated with a range of wavelengths that includes the selected wavelength. In this variation, the generated light may be tuned across the range of wavelengths and a DC signal from the photodetector may be demodulated in the control unit to yield a 2f signal that is analyzed to determine the absorbance at the selected wavelength. The methane concentration may be less than or equal to approximately 50 ppm and the selected wavelength may be in the range of about 1685 nm to 1700 nm. The methane concentration may be less than or equal to approximately 50 ppm and the selected wavelength may be one of approximately 1654 nm, approximately 1687 nm, approximately 1694 nm, and approximately 1697 nm. The methane concentration may be in the range of approximately 1% to approximately 5% and the selected wavelength may be in the range of about 1630 nm to 1660 nm. The methane concentration may be in a range of approximately 1% to 5%, and the selected wavelength may be one of approximately 1637.4 nm, 1640.4 nm, 1642.9 nm, 1645.5 nm, 1648.2 nm, 1650.9 nm, 1653.7 nm, and 1656.5 nm. The beam of light may be provided from a tunable diode laser than is tuned to provide a range of wavelengths comprising the selected wavelength. The gas mixture and the photodetector may be maintained at a constant temperature within a tolerance of approximately ±1° C.

The path length may be equal to or less than approximately 50 cm. Alternatively, the path length may be less than approximately 40 cm. The laser light source may be modulated based on a modulation signal provided by the control unit and the control unit may be configured to demodulate the direct current signal from the photodetector to generate a second harmonic signal that is analyzed to determine the intensity of light traversing the path length at the selected wavelength. The laser light source may be selected from a vertical cavity surface emitting laser, a horizontal cavity surface emitting laser, a quantum cascade laser, a distributed feedback laser, and a color center laser. A thermally controlled chamber may be provided that encloses one or more of the laser source, the photodetector, and the sample cell.

### DESCRIPTION OF THE DRAWINGS

This disclosure may be better understood upon reading the detailed description and by reference to the attached drawings, in which:

FIG. 1 is a process flow diagram illustrating a method of analyzing the methane concentration in a carbon dioxide gas mixture.

FIG. 2 is a schematic diagram showing an example of an absorption spectrometer;

FIG. 3 is a schematic diagram showing an example of a multipass absorption cell;

FIG. 4 is a chart that illustrates principles of wavelength modulation spectroscopy;

FIG. 5 is a chart showing an example of a DC signal and a 2f signal of wavelength modulation spectroscopy;

FIG. 6 is a block diagram of a measurement system;

FIG. 7 is a chart showing an example of a laser current drive signal;

FIG. 8 is a chart showing absorbance of gases in the wavelength range between about 1620 nm and 1680 nm; and

FIG. 9 is a chart showing absorbance of gases in the wavelength range between about 1620 nm and 1740 nm

### DETAILED DESCRIPTION

Absorption spectroscopy can be used to measure low concentrations of various trace gases in gas mixtures. In general, a light beam of suitable wavelength is passed through a sample of a gas that is contained within a sample cell. As light passes through the gas, some of its intensity is absorbed by trace gas molecules of compounds that absorb at that specific wavelength. The amount of light absorbed is dependent on the concentration (partial pressure) of compounds that absorb at the specific wavelength and can therefore be used to calculate the concentration. This arrangement is suitable when the background gas has no or very weak absorption features in the spectral region being used for the trace gas measurement.

Near infrared radiation generally lacks sufficient photon energy to induce absorption by electronic transitions such as those induced by ultraviolet radiation. Therefore, IR absorption is restricted to compounds with small energy differences in the possible vibrational and rotational states of the molecules. For a molecule to absorb IR radiation, the vibrations or rotations within a molecule must cause a net change in the dipole moment of the molecule. The alternating electrical field of the radiation interacts with fluctuations in the dipole moment of the molecule. The energy of the incident light radiation is
E=hv (I)
where E is the photon energy, h is Planck's constant and v is the frequency of the light. If E matches the energy necessary to excite a vibrational mode of a molecule, then radiation will be absorbed causing a change in the amplitude of this molecular vibration. The two main types of molecular motion, which includes relative motion between atoms making up the molecule, involve stretching and vibration of inter-atomic bonds.

Stretching transitions require moderate energies and are therefore quite useful to IR absorption spectroscopy. In stretching transitions, the inter-atomic distance changes along bond axes, and the resultant absorbance of IR by gas-phase molecules yield line spectra sufficiently spaced apart to allow detection. In liquids or solids, these lines broaden into a continuum due to molecular collisions and other interactions such that they cannot be measured by IR absorption spectroscopy.

The relative positions of atoms in molecules are not fixed, but are rather subject to a number of different vibrations relative to other atoms in the molecule. A specific molecular motion requires a corresponding quantum of activating photon energy. Therefore, an incident photon's energy must be of exactly the right wavelength to be absorbed into the molecule. Thus, if a gas containing a molecule that absorbs and vibrates at a given wavelength λ is illuminated by a beam of light of wavelength λ, some of the incident photons will be absorbed as it passes through the gas. This absorbance A_{i,λ} is calculated from the beam power incident on the sample P₀ and the beam power passing through the sample P as follows:
A_{i,λ}=−ln(P/P₀) (2)

In accordance with Beer-Lambert's Law, the absorbance A_{i,λ} due to a specific gas-phase compound i at the incident wavelength λ is directly proportional to its concentration Cᵢ in the cell:
A_{i,λ}=Cᵢε_{i,λ}L (3)
where ε_{i,λ} is the extinction coefficient for the compound at the incident wavelength, and L is the path length of the absorption/sample cell.

An analyzer used in connection with the subject matter disclosed here can be used to make measurements of methane in gas mixtures containing a substantial background concentration of carbon dioxide, up to and including a mixture of only methane in carbon dioxide. In general, such an analyzer includes a source of incident light, such as a laser, one or more detectors with sensitivity in the wavelength range of the light source, and one or more absorption cells, each arranged such that the gas provides a path length L though which a beam from the light source passes before reaching the detector. Control electronics, such as a microprocessor, and user accessible input/output channels can also be included. The following is a general description of various examples of such devices and their operation.

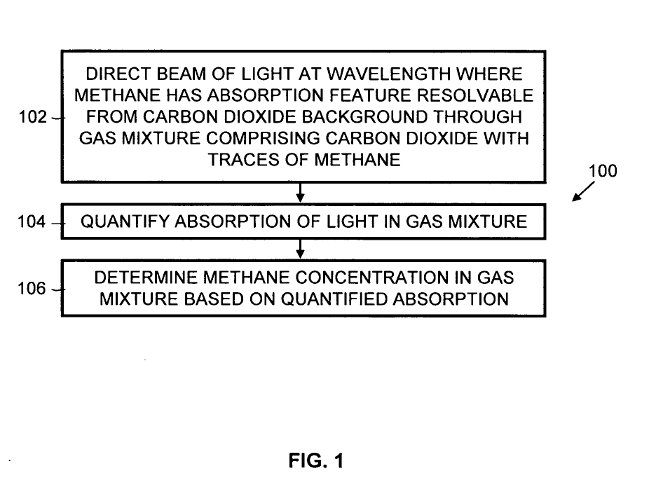

FIG. 1 shows a flow chart 100 of a method of analyzing the methane concentration is a gas mixture containing carbon dioxide. In general, a beam of light is directed through a gas mixture at 102. The light includes at least one wavelength at which methane has an absorption feature that is resolvable from a carbon dioxide background. The gas mixture can have a methane concentration of less than or equal to approximately 5%. Alternatively, the methane concentration can be greater than 5%. At 104, absorption of light in the gas mixture is quantified over an absorption path length of less than or equal to approximately 50 cm. The methane concentration in the gas mixture can be determined at 106 using the quantified absorption and a calibration function. The measurement of transmitted intensity can be performed with a photodetector such as described below. The recording of the absorption spectrum can be performed with a data analysis device, such as for example a microprocessor.

In one implementation, a sample of a gas containing methane and carbon dioxide is illuminated by a laser light source that emits light either in a continuous or a pulsed beam. The light source can be a laser such as a tunable diode laser (TDL) or alternatively a fixed wavelength laser light source operating at a specific wavelength chosen to have detectable methane absorption without interference from high concentrations of carbon dioxide. The calibration function can be determined based on analysis of one or more samples of gas containing methane and/or carbon at known concentrations. The samples are analyzed using the analyzer to be used in the above method. Absorption of light at the selected wavelength is quantified for each sample, and a fitting function is applied to relate methane concentration to measured absorption. The calibration function can be a linear relationship or it can alternatively be a more complex mathematical function.

Examples of tunable lasers that can be used are the distributed feedback laser (DFB), the vertical cavity surface emitting laser (VCSEL), and the horizontal cavity surface emitting laser (HCSEL). These lasers can be direct emitters or fiber coupled. Quantum cascade lasers can also be utilized as can other lasers capable of producing a beam of incident light in the desired wavelength range. Additional detail about these types of lasers is available in co-pending U.S. patent application Ser. No. 11/715,599, the disclosure of which is hereby incorporated by reference in its entirety.

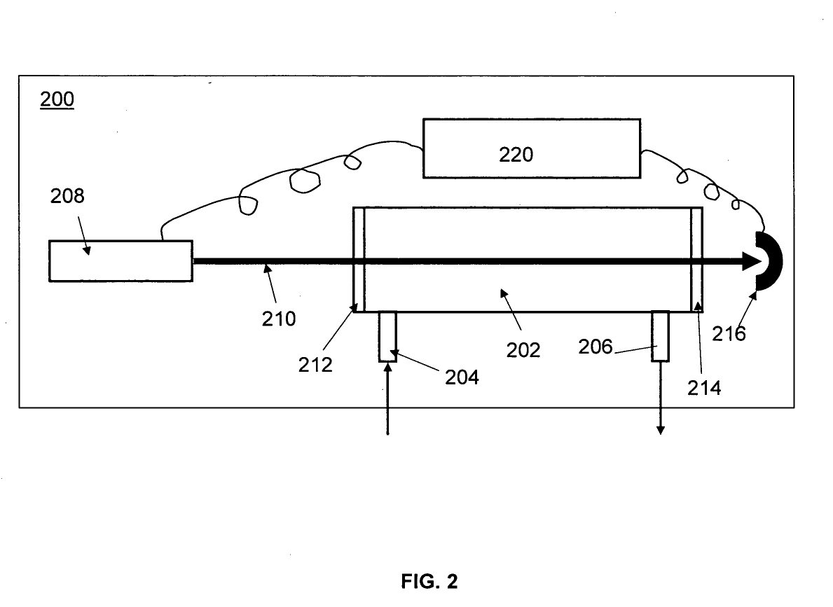

An illustrative implementation of an analyzer as disclosed herein is depicted schematically in FIG. 2, which shows an analyzer 200 that implements various aspects of the current subject matter. In this implementation, a gas sample is contained within a sample cell 202. The gas sample can be directed into the sample cell 202 via an inlet 204 and flushed from the sample cell 202 via an outlet 206. In some variations, the inlet 204 and the outlet 206 can include valves that can seal the inner volume of the sample cell 202 to obtain a static measurement of a fixed volume of gas. If there are no inlet and outlet valves, or if the inlet and outlet valves are open, the system can be used in a continuous or semi-continuous flow mode, such as for example to continuously or semi-continuously monitor the concentration of a target analyte in a flowing gas stream. For continuous or semi-continuous operation, all or part of a gas stream is directed into the sample cell 202 via the inlet 204 and flushed out of the sample cell 202 via the outlet 206 by the flow of the gas. Flow through the sample cell 202 can be caused by a pressure differential created by a pump or some other mechanism.

A light source 208 that provides light with at least a wavelength where methane absorption can be resolved from a carbon dioxide background generates a continuous or pulsed beam 210 that is directed through the gas volume of the sample cell 202. In the example shown in FIG. 2, the sample cell includes an windows 212 and 214 that allow the light beam 210 to enter and exit the cell. The light beam 210 is directed onto a photodetector or other device for quantifying the intensity of incident light 216 as the light beam exits the sample cell 202. The photodetector 216 is electronically coupled to a control unit 220 that can optionally also be electronically coupled to the light source 208 as shown in FIG. 2. The control unit 220 can include one or more processors coupled to a memory that stores instructions in computer readable code. When executed on the processor or processors, the instructions can implement a method, such as for example that described above, to analyze the absorption at the second or reference wavelength to infer and compensate for the absorption at the first or target wavelength that is due to the background analyte. Once the absorption at the first or target wavelength is so compensated, the control unit 220 can calculate the target analyte concentration.

If the control unit 220 is electronically connected to the light source 208, it can optionally control the light source. For example, if the light source 208 is a tunable diode laser, such as one of those described in U.S. patent application Ser. No. 11/715,599, the control unit can control the scan rate and also interpret the direct voltage measurements by the photodetector 216 to convert them to modulated 2f values. The control unit can also adjust the modulation amplitude as necessary to improve spectral resolution.

Other analyzer configurations besides that shown in FIG. 2 are possible, including but not limited to those described in co-pending U.S. patent application Ser. No. 11/715,599. The sample cell 202 can be a single pass design in which the light beam 210 from the light source 208 passes once through the gas volume of the sample cell 202 before exiting the sample cell 202. In this configuration, the optical path length is effectively the length of the sample cell 202. It is also possible to use one or more mirrors that reflect the light beam 210 such that it passes through the sample volume more than once before exiting the sample cell 202. A Herriot cell (described in full detail in co-pending U.S. patent application Ser. No. 11/715,599, in which the light beam 210 is reflected between two spherical mirrors numerous times to create a very long optical path length, can also be used. The optical path length can be selected based on the strength of the absorption features being used in a measurement and the concentration of the gases being analyzed.

The path length of the sample cell can be varied depending on the strength of the specific absorption line of interest or the magnitude of the difference between the absorption line of interest and interfering absorption lines from other gas species present. A cell of insufficient length can not provide sufficient sensitivity while one of excessive length can absorb the entirety of the incident light such that no measurable signal reaches the detector (a situation called saturation).

In some cases, the concentration of methane in a carbon dioxide-rich gas mixture can be very small or not readily distinguishable from other components present in the gas. In such cases, the length of the cell can be increased to increase the sensitivity of the measurement. As equation 3 states, A_{i,λ} is directly proportional to the path length L over which the laser beam traverses the gas mixture. Thus, a cell that is twice as long will absorb twice as much light etc. Therefore, in some implementations of the analyzers described here, sample cells can be employed having path lengths on the order of many meters or even thousands of meters.

To achieve longer optical path lengths without the use of extremely long sample cells, sample cell configurations within the scope of this disclosure can also include the use of one or more mirrors to reflect the beam such that the beam passes through the sample contained in the sample cell two or more times. In such a multipass configuration, the beam can enter and exit the cell through the same window or through different windows. In some implementations, windowless sample cell configurations can be utilized in which, for example, the laser source and/or the photodetector are contained within the sample cell.

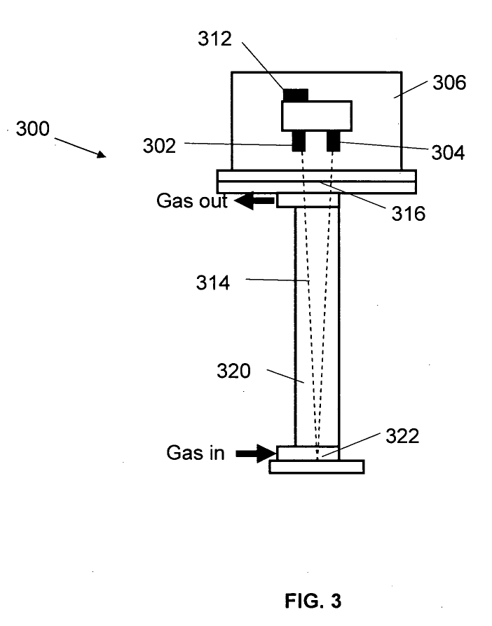

One example of such a multipass sample cell configuration is shown in FIG. 3, which depicts a two-pass absorption cell and laser/detector head 300. A laser 302 and photodetector 304 are positioned in an optical head 306 mounted to a baseplate 310 whose temperature is controlled by a thermoelectric cooler (TEC) 312. The incident laser light 314 is directed out of the optical head 306 through a window 316 into the sample cell 320. The light travels the length of the sample cell 320 twice as it is reflected at the far end of the cell by a flat mirror 322. The returning light is transmitted back through the window 316 and impinges on the photodetector 304. The analyzer shown in FIG. 2 can be modified to incorporate a multipass detector head as shown in FIG. 3.

The light source used for the absorption measurements disclosed can emit in the infrared (for example in a wavelength range of approximately 800 to 10,000 nm). The analyzer can utilize a laser whose spectral bandwidth is much narrower than the bandwidth of the absorption lines of interest. Such an arrangement allows for single line absorption spectroscopy in which it is not necessary to scan the entire width of the absorption line or even the peak absorption feature of the line. The wavelength of the laser can be chosen to be one at which there is a resolvable difference in the relative absorbance of water molecules and the other components of the gas to be measured. In one implementation, the laser frequency can be scanned (tuned) back and forth across the chosen absorption wavelength while a photodetector positioned at the opposite end of the beam path length quantifies the light intensity transmitted through the sample as a function of wavelength.

With the laser absorption spectrometers described herein, the tunable laser wavelength can be varied by changing the injection current while keeping the laser temperature constant. The temperature can be controlled by placing the laser in intimate contact with a thermoelectric cooler (Peltier cooler) whose temperature is measured with a thermistor and controlled by a feedback circuit. The control unit of a device, system, or apparatus as described herein can provide process control functions to regulate the system temperature.

In some implementations, an absorption spectrometer system can employ a harmonic spectroscopy technique in connection with its TDL light source. Harmonic spectroscopy as used in the disclosed subject matter involves the modulation of the TDL laser (DFB or VCSEL) wavelength at a high frequency (kHz-MHz) and the detection of the signal at a multiple of the modulation frequency. If the detection is performed at twice the modulation frequency, the term second harmonic or “2f” spectroscopy is used. Advantages to this technique include the minimization of 1/f noise, and the removal of the sloping baseline that is present on TDL spectra (due to the fact that the laser output power increases as the laser injection current increases, and changing the laser injection current is how the laser is tuned).

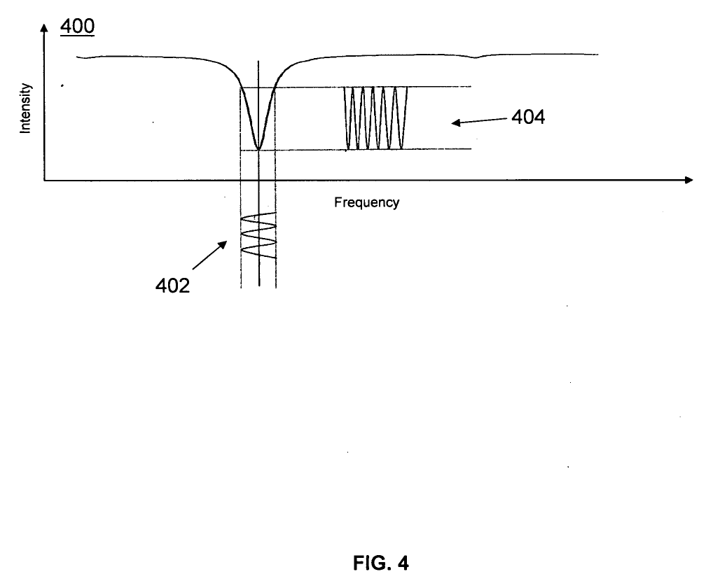

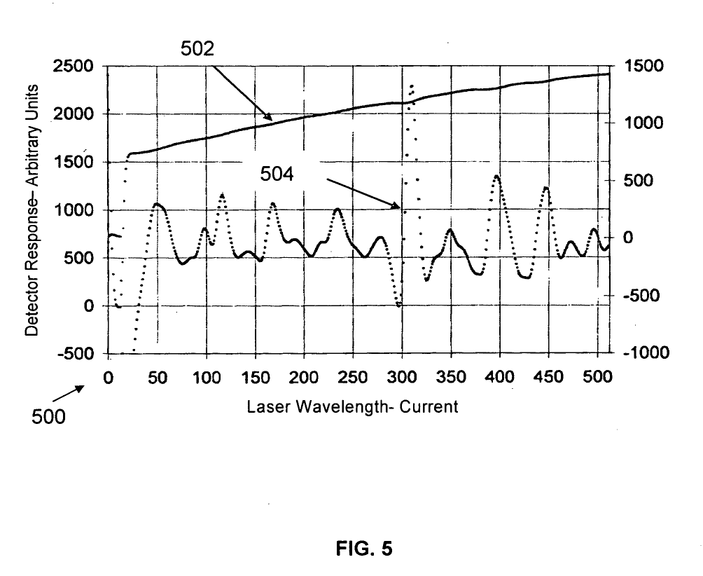

FIG. 4 shows an example of a laser scan 400 for use in harmonic spectroscopy. A combination of a slow ramp and a fast sinusoidal modulation of the wavelength 402 is used to drive the diode laser. The photodetector receives this modulated intensity signal 404. The N^{th} harmonic component is resolved by demodulating the received signal. Detection using the signal at the second harmonic (2f) can be used. The 2f lineshape is symmetric and peaks at line center due to the nature of even function. Additionally, the second harmonic (2f) provides the strongest signal of the even-numbered harmonics. FIG. 5 presents a chart 500 of a typical direct current laser intensity signal 502 and a demodulated 2f lineshape 504 vs. frequency. By shifting detection to higher frequency, 2f spectroscopy can significantly reduce 1/f noise thus provides a substantial sensitivity enhancement compared to direct absorption methods.

In another implementation, direct absorption spectroscopy can be used. In this implementation, the laser frequency is tuned over the selected absorption transition and the zero-absorption baseline can be obtained by fitting the regions outside the absorption line to a low-order polynomial. The integrated absorbance is directly proportional to the concentrations of absorbing species in the laser path length as well as the line strength of the transition. The absolute species concentration can be obtained without any calibration

Photodetectors used in the disclosed absorption spectrometers depend on the specific wavelengths of the lasers and absorption lines to be measured. One photodetector is an indium gallium arsenide (InGaAs) photodiode sensitive to light in the 1200 to 2600 nm wavelength region. For longer wavelengths, an indium arsenide photodiode, sensitive for wavelengths up to approximately 3.6 μm, can be used. Alternatively, indium antimonide detectors are currently available for wavelengths as long as approximately 5.5 μm. Both of the indium devices operate in a photovoltaic mode and do not require a bias current for operation. These photodetectors, which lack low frequency noise, are advantageous for DC or low frequency applications. Such detectors are also advantageous for high speed pulse laser detection, making them particularly useful in trace gas absorption spectroscopy. Other photodetectors, such as for example indium arsenide (InAs), silicon (Si), or germanium (Ge) photodiodes and mercury-cadmium-telluride (MCT) and lead-sulfide (PbS) detectors, may also be used.

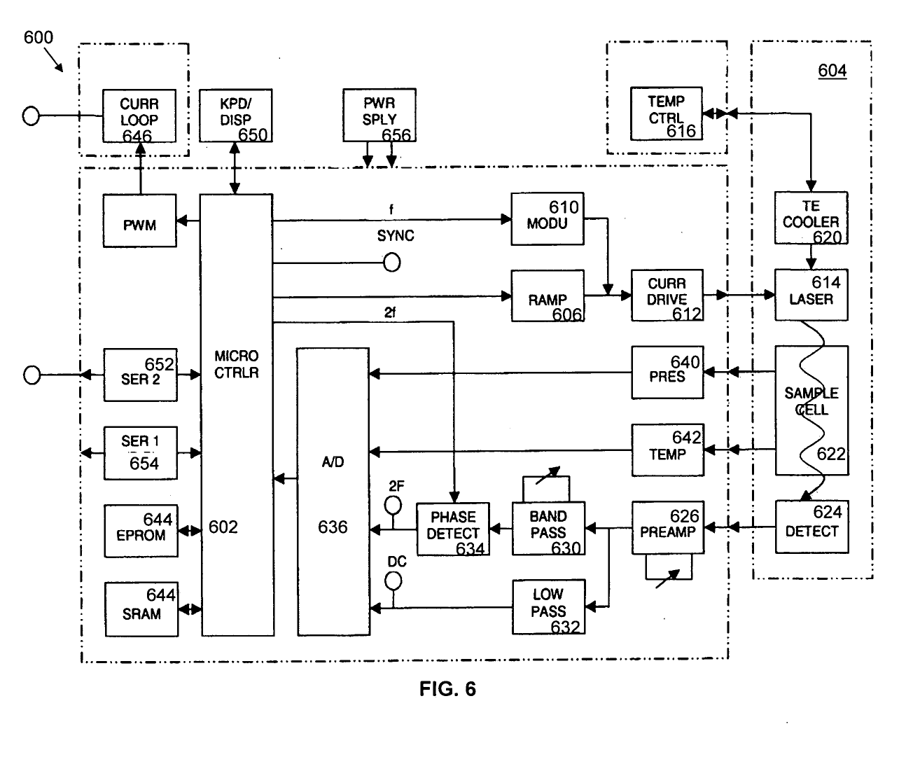

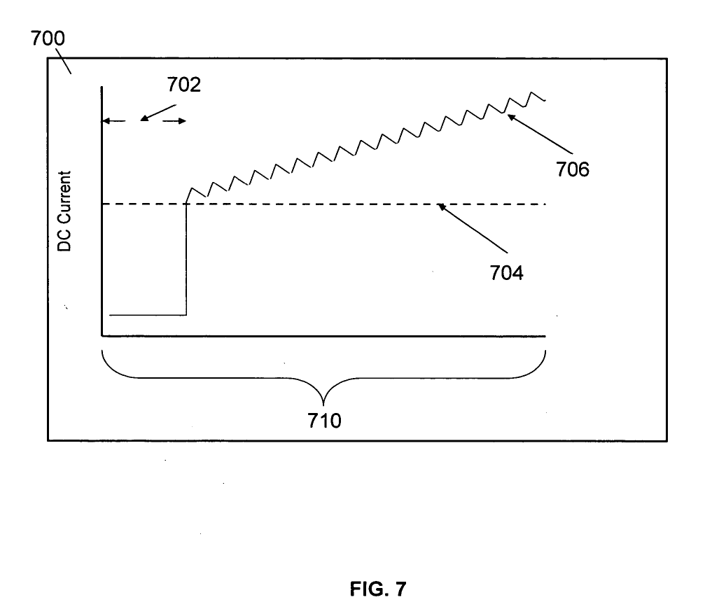

FIG. 6 is a diagram of a sensor system 600 that includes a control and data processing loop system with a microprocessor 602 in communication with a spectrometer 604. On command, a signal is generated by the microprocessor 602 in the form of a rectangular pulse. This pulse is generated periodically. In one implementation, a 263 msec wide pulse is generated every 0.25 seconds. Other pulse widths and generation frequencies can be utilized. Each pulse is directed toward a ramp generator 606 that creates a DC signal, an example of which is shown diagrammatically in FIG. 7. In addition to the ramp signal, a modulating sine wave, at for example 7.5 KHz, can be imposed on the current ramp by a modulator 610 for later use in small signal detection. This combined signal is directed to the laser current driver 612 and on to the laser 614 itself.

In this implementation, the laser temperature is held constant by a temperature controller board 616 and the current varied for tuning the laser wavelength. The temperature control loop uses a thermistor (not shown) located close to the laser 614 as the temperature input and a thermoelectric cooler 620 mounted as close (thermally) to the laser 614 as possible. TECs and thermistors can be positioned either directly adjacent to the laser diode or externally to the laser diode enclosure. The temperature controller 616 can be used to set the exact laser wavelength such that variation of the driving current can provide the tuning range which can, for example, be in the range of approximately ±0.3 cm⁻¹.

At the beginning of each measurement cycle, the current is held to zero to read the signal produced by the photodetector without laser input and thereby provide the zero for that measurement cycle. This zero can vary a small amount due to slight changes in the detector dark current and the electronic noise so it is advantageous to measure it during each detector cycle. Following determination of the zero, the current is rapidly increased to the laser threshold current. This current is then increased over the remainder of the cycle until the peak current is reached. The beam created from this signal is directed through the sample cell 622 and onto the detector 624 which can be a photodiode array or other comparable detector. The output current from the detector is first amplified by a preamplifier 626. The output of the preamplifier is split and sent to a bandpass filter 630 and a lowpass filter 632. The bandpass filter 630 is a narrowband filter that singles out the 2f signal at 15 KHz and directs it to a lock-in amplifier 634 whose reference is set at 15 KHz from a signal provided by the microprocessor. The lock-in amplifier 634 further amplifies the signal and directs it to an A-D board 636 and back into the microprocessor 602. The lowpass filter 632 provides the detector output except the 2f signal. This signal provides the microprocessor 602 with the zero for the system and is also a diagnostic tool.

The signal is developed and recorded by the microprocessor 602 for each cycle of the analyzer. The processor determines the concentration of the gas sample of interest by computing the absorbance of the gas as a ratio between the zero and the measured value of absorbance at the peak of the absorbance line. The absorbance is a function of the gas pressure and temperature in the sample cell 622 which are measured by appropriate means 642 and 644, respectively, whose outputs are supplied to the A/D board 636. The absorbance can be adjusted by a pressure/temperature calibration matrix stored in the microprocessor memory 644. This matrix is developed on an analyzer-by-analyzer basis. Alternatively, one or more corrective calculations can be performed based on measured temperature and pressure in the sample cell.

Once the corrected absorbance value is determined, the concentration can be computed using equation 3. In one implementation, this concentration can be converted into units of, for example lbs/mmscf, averaged four times, and sent to the outputs once per second. Outputs that can be included in this system are a 4-20 mA current loop 646, a visual display 650 and RS-232 or comparable serial ports 652 and 654. Power for the system is provided by an appropriately chosen power supply 656.

The chart of laser current vs. time 700 shown in FIG. 7 illustrates an example of the laser pulse profile that may be used in the disclosed analyzers. For each pulse cycle, A dynamic zero measurement is made during an initial period 702 when the laser current is well below the lasing threshold 704. Then, the laser current is ramped rapidly to at or above the lasing threshold 704, and a modulated laser tuning ramp with an alternating current voltage 706 is added to facilitate the 2f demodulation calculations as described above. At the end of the pulse cycle 710, the process is repeated. In one example, the pulse cycle last approximately 263 milliseconds. Other cycle periods are within the scope of this disclosure.

The specific absorption transitions for measurement of CH₄ in various background gases which contains CO₂ are summarized in Table 1. However, other wavelength ranges may be utilized provided that methane molecules absorb light at a greater level than do background gas molecules. Selection of an individual absorption line may be completed using a Figure of Merit (FOM), which is defined as the absorption of 1 ppmv methane divided by the total background absorption. All methane lines satisfying an FOM of approximately greater than or equal to 1×10⁻⁶ are suitable for sensitive detection of methane.

TABLE 1
Illustrative absorption transitions for measurement of methane
1637.7 nm
1640.4 nm
1642.9 nm
1645.5 nm
1648.2 nm
1650.9 nm
1653.7 nm
1656.5 nm
1666.2 nm
1674.4 nm
1677.6 nm
1684.0 nm
2261.7 nm
2275.4 nm
2284.0 nm
2289.9 nm
2307.5 nm
2317.1 nm
2350.5 nm
2351.0 nm
2355.8 nm
2358.9 nm
2370.4 nm
2371.7 nm

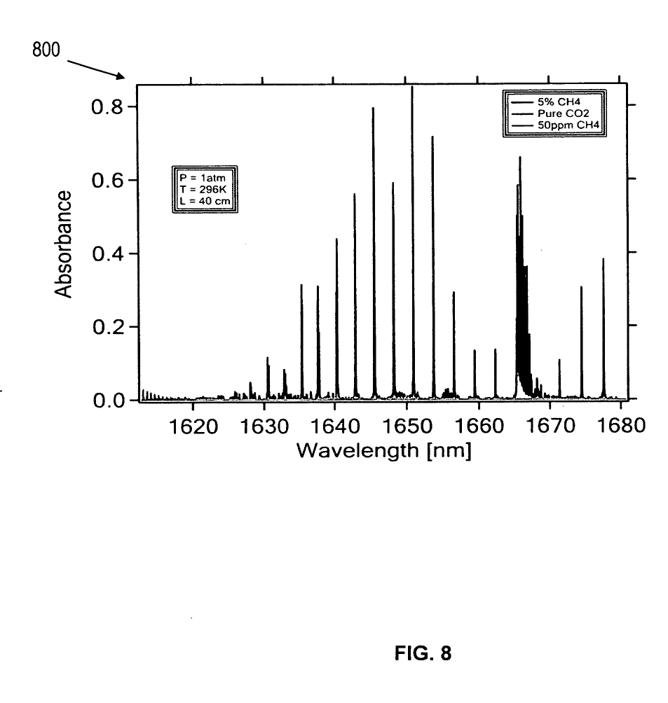

Spectrometers described herein can be used to accurately and repeatedly measure low levels, for example at concentrations of less than approximately 1% or alternatively less than approximately 5% of methane in carbon dioxide. Measurement of concentrations of methane in carbon dioxide gas in a range of approximately 1% to 5% methane can be performed at a wavelength of approximately 1645 nm using TDLAS spectroscopy with an absorption path length that is less than or equal to approximately 40 cm and at approximately atmospheric pressure. Specific methan transitions near 1645 nm are listed in table 1. As can be seen from FIG. 8, which shows a chart 800 of absorbance vs. wavelength for methane and carbon dioxide, potential measurement wavelengths that have been discovered in the range between approximately 1630 and 1660 nm are listed in Table 1. These wavelengths can also be used for measurement of methane in carbon dioxide at the methane concentrations noted above. A TDLAS spectrometer with a single-beam arrangement can be employed as described above. Here, the sample gas is flowed through a sample cell while the laser wavelength is altered using the techniques described above. This wavelength scan across the absorption line of interest and directed at a detector which detects a “2f” signal. Similar spectroscopic techniques used in the past have not been successful in measuring methane (CH₄) in carbon dioxide (CO₂) to these levels of accuracy. This is because a suitable absorption line had not been determined.

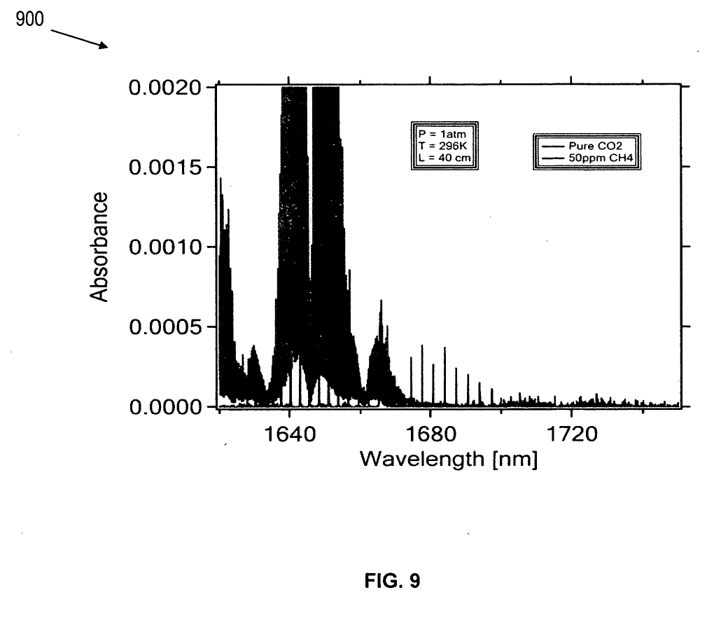

It is also possible to quantify methane in carbon dioxide at concentrations less than or equal to approximately 50 ppm. The technique has been found to be successful at wavelengths between 1685 and 1700 nm with an absorption path length of less than or equal to approximately 50 cm as shown in the absorbance chart 900 in FIG. 9. Specific lines that can be used include, but are not limited to those listed in Table 1 in this range. A tunable laser operating at a wavelength of approximately 1691 nm can be used to emit at the strongest absorption line (1684.0 nm) of methane without CO₂ interference. The absorption cell can be maintained to a single temperature to within ±1° C. and used at 1 atmosphere. Temperature control can be accomplished by placing the spectrometer in a thermally controlled enclosure who's interior is insulated and temperature is held above 30° C. in conditions where the environment can vary from −15° C. to +60° C.

The subject matter described herein can be embodied in systems, apparatus, methods, and/or articles depending on the desired configuration. In particular, various aspects of the subject matter described herein can be realized in digital electronic circuitry, integrated circuitry, specially designed application specific integrated circuits (ASICs), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the term “machine-readable medium” refers to any computer program product, apparatus and/or device (e.g., magnetic discs, optical disks, memory, programmable logic devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Although a few variations have been described in detail above, other modifications, additions, and implementations are possible are within the scope and spirit of the disclosed subject matter. Features implementing functions can also be physically located at various positions, including being distributed such that portions of functions are implemented at different physical locations. Further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not require the particular order shown, or sequential order, to achieve desirable results.

What is claimed:

1. A method of detecting trace amounts of methane in carbon dioxide backgrounds, comprising:
directing a beam of light at a selected wavelength through a gas mixture comprising carbon dioxide and methane, the selected wavelength coinciding with a methane absorption feature that is resolvable from an absorption background due to carbon dioxide;
quantifying an absorption at the selected wavelength in the gas mixture; and
determining a methane concentration in the gas mixture based on the quantified absorption.

2. A method as in claim 1, wherein the gas mixture is contained within a sample cell that provides the path length.

3. A method as in claim 1, wherein the absorption at the selected wavelength is quantified with a photodetector that provides a detector output signal to a microprocessor.

4. A method as in claim 2, further comprising:
generating light with a range of wavelengths, the range of wavelengths comprising the selected wavelength;
tuning the generated light across the range of wavelengths; and
converting a DC signal from a photodetector that the light beam impinges upon after traversing the gas mixture to a second harmonic signal by demodulating the DC signal; and
analyzing the second harmonic signal to determine the methane concentration.

5. A method as in claim 1, wherein the methane concentration is less than or equal to approximately 50 ppm and the selected wavelength is in the range of about 1685 nm to 1700 nm.

6. A method as in claim 1, wherein the methane concentration is less than or equal to approximately 50 ppm and the selected wavelength is one of approximately 1654 nm, approximately 1687 nm, approximately 1694 nm, and approximately 1697 nm.

7. A method as in claim 1, wherein the methane concentration is in the range of approximately 1% to approximately 5% and the selected wavelength is in the range of about 1630 nm to 1660 nm.

8. A method as in claim 1, wherein the methane concentration is in a range of approximately 1% to 5%, and the selected wavelength is one of approximately 1637.4 nm, 1640.4 nm, 1642.9 nm, 1645.5 nm, 1648.2 nm, 1650.9 nm, 1653.7 nm, and 1656.5 nm.

9. A method as in claim 1, further comprising providing the beam of light from a tunable diode laser than is tuned to provide a range of wavelengths comprising the selected wavelength.

10. A method as in claim 1, further comprising maintaining the gas mixture and the photodetector at a constant temperature within a tolerance of approximately ±1° C.

11. An apparatus comprising:
a laser light source that emits at a selected wavelength that coincides with a methane absorption feature that is resolvable from an absorption background due to carbon dioxide;
a sample cell to contain a gas mixture containing methane and carbon dioxide with a methane concentration of less than or equal to approximately 5%, the sample cell providing a path length through the gas mixture of less than or equal to approximately 50 cm;
a photodetector positioned to quantify an intensity of light traversing the path length and to output a direct current data signal based on the quantified intensity; and
a microprocessor configured to receive and interpret the direct current signal from the photodetector and to determine the methane concentration in the gas mixture based on the direct current data signal and a calibration function.

12. An apparatus as in claim 11, wherein the methane concentration is less than or equal to approximately 50 ppm and the selected wavelength is in the range of about 1685 nm to 1700 nm.

13. An apparatus as in claim 11, wherein the methane concentration is less than or equal to approximately 50 ppm and the selected wavelength is one of approximately 1654 nm, approximately 1687 nm, approximately 1694 nm, and approximately 1697 nm.

14. An apparatus as in claim 11, wherein the methane concentration is in the range of approximately 1% to approximately 5% and the selected wavelength is in the range of about 1630 nm to 1660 nm.

15. An apparatus as in claim 11, wherein the methane concentration is in a range of approximately 1% to 5%, and the selected wavelength is one of approximately 1632 nm, approximately 1637.4 nm, 1640.4 nm, 1642.9 nm, 1645.5 nm, 1648.2 nm, 1650.9 nm, 1653.7 nm, and 1656.5 nm.

16. An apparatus as in claim 11, wherein the laser light source is a tunable diode laser.

17. An apparatus as in claim 16, wherein the laser light source is modulated based on a modulation signal provided by the microprocessor and wherein the microprocessor is configured to demodulate the direct current signal from the photodetector to generate a second harmonic signal that is analyzed to determine the intensity of light traversing the path length at the selected wavelength.

18. An apparatus as in claim 16, wherein the laser light source is selected from a vertical cavity surface emitting laser, a horizontal cavity surface emitting laser, a quantum cascade laser, a distributed feedback laser, and a color center laser.

19. An apparatus as in claim 11, further comprising a thermally controlled chamber that encloses one or more of the laser source, the photodetector, and the sample cell.

20. A method of detecting trace amounts of methane in carbon dioxide backgrounds, comprising:
directing a beam of light at a wavelength in the range of approximately 1630 nm to 100 nm through a gas mixture comprising carbon dioxide and less than approximately 5% methane, the selected wavelength coinciding with a methane absorption feature that is resolvable from an absorption background due to carbon dioxide, the beam of light being provided by a tunable diode laser;
quantifying an absorption at the selected wavelength in the gas mixture over a path length of less than or equal to approximately 50 cm; and
determining a methane concentration in the gas mixture based on the quantified absorption and a calibration function.
